(19) United States
(12) Patent Application Publication
(10) Pub. No.: US 2015/0164920 A1
(43) Pub. Date: Jun. 18, 2015

(54) CONTROLLED RELEASE FORMULATION COMPRISING MESALAMINE

(71) Applicant: LABORATORIOS DEL DR. ESTEVE S.A., Barcelona (ES)

(72) Inventors: Luis Soler Ranzani, Barcelona (ES); Albert Falivene Aldea, Barcelona (ES); Gemma Casadevall Pujals, Barcelona (ES)

(73) Assignee: LABORATORIOS DEL DR. ESTEVE S.A., Barcelona (ES)

(21) Appl. No.: 14/388,308

(22) PCT Filed: Mar. 27, 2013
(86) PCT No.: PCT/EP2013/056460

§ 371 Date: Sep. 26, 2014

(30) Foreign Application Priority Data
Mar. 30, 2012 (EP) 12384003.5

(51) Int. Cl. A61K 31/606; A61K 9/28; A61K 9/50

(52) U.S. Cl. CPC A61K 31/606; A61K 9/5015; A61K 9/5047; A61K 9/2866

(57) ABSTRACT

The present invention refers to a controlled release pharmaceutical formulation in solid form comprising Mesalamine. The invention also contemplates the preparation of the said formulation and its use for the treatment of patients suffering from inflammatory bowel disease (IBD).

### FIELD OF THE INVENTION

The present invention refers to a controlled release pharmaceutical formulation in solid form comprising Mesalamine. The invention also contemplates the preparation of the said formulation and its use for the treatment of a patient suffering from inflammatory bowel disease.

### BACKGROUND OF THE INVENTION

Gastrointestinal conditions are a significant worldwide health problem. Inflammatory bowel disease (IBD), which encompasses a range of diseases including Crohn's disease (CD) and ulcerative colitis (UC), affects nearly 1 million people in the USA each year.

Although UC and CD are different conditions, the same drugs are commonly used to treat both. Drugs commonly used in their treatment include steroids, cytokines, immunomodulating agents, anti-tumor necrosis factor agents and antibodies and also anti-inflammatory agents. The First-line therapy for patients with mild to moderate IBD are 5-aminosalicylic acid derivatives which comprise: a) Mesalamine or (5-ASA) including oral and rectal Mesalamine formulations, b) 4-ASA also known as para-aminosalicylic acid and c) oral pro-drugs (sulfasalazine [5-aminosalicylic acid linked to sulfapyridine], olsalazine [5-aminosalicylic acid dimer] and basalazide [5-aminosalicylic acid linked to 4-aminobenzoyl-β-alanine]).

Due to its good tolerability in comparison with the rest of its prodrugs, Mesalamine, has been established as a common treatment for IBD and is widely prescribed and used for this purpose.

Existing oral 5-ASA-based therapies fall into two main categories. One involves the use of pharmaceutical dosage forms on modified-release formulations, the other is pro-drug based. In relation to dosage form based approaches, various modified release pharmaceutical compositions have been thought and described. Both extended/sustained release formulations and delayed release formulations have been developed, with the intent of limiting 5-ASA release in the upper gut and concentrating its release in the distal gut. To achieve this goal there are two main approaches: a) one of them relies on formulations which releases Mesalamine continuously through the entire gastrointestinal tract and b) the other relies on a pH-dependent coating which targets the release of Mesalamine in a specific part of the gastrointestinal tract.

An example of the first type of approaches is the marketed product PENTASA® which is a sustained release formulation releasing 5-ASA continuously, with approximately 50% released in the small intestine and 50% in the large intestine. In the approved leaflet of PENTASA®, 20-30% systemic absorption is reported.

Patent U.S. Pat. No. 4,880,794 describes sustained release granules of Mesalamine coated with ethyl cellulose. The resulting Mesalamine coated granules are sprayed with a solution of ethylcellulose in acetone. This formulation presents the drawback of using organic solvents in the preparation of the formulation which is not preferred for environmental reasons.

Patent EP 871 434 B2 deals with a modified release composition comprised of individually granules which are coated with a cellulose polymer and which target the proximal, the mid and the distal small intestine, the caecum, the ascending colon, the transverse, the descending and the sigmoid colon and/or the rectum. According to the authors, systemic effects and other adverse effects due to the 5-ASA are negligible with the described formulation. Therefore, this formulation does not show systemic 5-ASA effects.

Patent EP 977 557 B1 describes an oral pellet formulation having controlled release profile comprising an active core wherein Mesalamine is present in a non gel-forming polymer and enteric coating. The enteric coating protects the formulation from the acidic gastric juice of the stomach and targets its absorption on the intestine. No release of Mesalamine is taken place in the stomach.

Patents EP 1 198 226 B1 and its divisional EP 1 287 822 B1 describe a controlled release formulation of Mesalamine which comprises an inner lipophilic matrix in which the active ingredient is partly dispersed and an outer hydrophilic matrix in which the active ingredient is dispersed. The composition releases no more than 30% within the first hour, no more than 50% within two hours, no more than 70% within four hours, no more than 90% within eight hours. The composition may optionally be coated with a gastro-resistant film. Therefore, due to the lag time of permanence in the stomach and partly in the intestine, no drug is released during that period of time. The preparation of this composition presents the disadvantage of melting the active ingredient with the lipophilic excipients to prepare the lipophilic matrix. In general, with melting techniques, the drug is exposed to high temperature, thus, they are not preferred since they may alter or degrade the drug substance.

U.S. Pat. No. 5,310,558 describes a programmed release pharmaceutical dosage form comprising a core, containing the active ingredient, coated by a hydrophobic layer. Such dosage form releases the active ingredient after a pre-established no-release interval. According to the teachings of this patent application after the programmed time interval the drug is released rapidly if the core is a prompt release preparation or is released slowly if the core is a controlled release preparation. This second alternative is particularly useful as far as drugs for the specific treatment of the colon like Mesalamine. Therefore, no release of Mesalamine is taken place in the stomach.

It is well known that patients suffering from IBD will develop extraintestinal disease manifestations or complications. Rheumatic manifestations of IBD include peripheral arthritis and axial involvement, including sacroilitis, and they are present in up to 62% of patients with IBD. The management of these rheumatic manifestations of IBD consists of physical therapy in combination with local injection of corticosteroids and nonsteroidal anti-inflammatory drugs. Sulfasalazine, methotrexate, azathioprine, cyclosporine and leflunomide should be used for selected indications (World J Gastroenterol 2009; 15 (44), 5517).

In view of the foregoing, there remains a need in the art for providing pharmaceutical formulations of Mesalamine that may be used both to treat IBD and its rheumatic manifestations.

### BRIEF DESCRIPTION OF THE INVENTION

The inventors of the present invention have surprisingly found that a pharmaceutical composition of Mesalamine comprising a specific mixture of a hydrophilic polymer and a waxy material allows the control of the drug release without affecting the drug stability (both chemical and physical) and achieves a degree of release of the Mesalamine in the acidic environment of the stomach. In contrast to the art, in the present invention, the release of the active ingredient can be controlled to modify the percentage of Mesalamine released in the stomach, thus allowing a degree of systemic absorption and, at the same time, ensuring an effective amount of Mesalamine released in the gastrointestinal tract. The Mesalamine absorbed systemically can perform activity outside the gastrointestinal system which provides a comfortable therapeutic alternative for IBD patients.

In particular, one aspect of the present invention relates to a controlled release oral formulation in solid form comprising:

a) an immediate release inner core comprising Mesalamine and a pharmaceutically acceptable excipient, and

b) a controlled release layer comprising a mixture of a waxy material and a hydrophilic polymer, said layer being directly disposed onto the inner core

This formulation offers advantages with respect to other formulations known in the art. For example, the formulation of the invention provides the delivery of effective concentrations of Mesalamine through the entire gastrointestinal tract being appropriate for the treatment of IBD. At the same time, the inventors have developed the formulation to be able to deliver an amount of Mesalamine in acidic pH which may perform a systemic anti-inflammatory action. Therefore, the formulation of the invention presents the advantage of providing an alternative therapy for the treatment of patients suffering IBD, or likely to suffer IBD, as well as providing the management of initial and non severe rheumatic manifestations of IBD. Thus, the use of the formulation of the invention may delay or reduce the common treatment of rheumatic IBD patients based on a combination of local injection of corticosteroids and nonsteroidal anti-inflammatory drugs.

In a further embodiment, the formulation is free from enteric coating.

In particular embodiments of the invention, the formulation in solid form is a particle, granule, pellet, minitablet or tablet.

In a second aspect, the invention relates to a tablet comprising the controlled release formulation of the invention. Typically, the tablet comprises a plurality of the above said particles, granules, pellets, minitablets or tablets.

In a further aspect, the invention relates to a capsule comprising the controlled release formulation of the invention. Typically, the capsule comprises a plurality of the above said particles, granules, pellets, minitablets or tablets.

In another aspect, the invention relates to a sachet or stick pack or a pouch comprising the controlled release formulation of the invention. Typically, the sachet, stick pack or pouch comprises a plurality of the above said particles, granules, pellets, minitablets or tablets.

Another aspect of the invention relates to the method for the preparation of the said formulation.

In a further aspect, the invention refers to the formulation of the invention for use as a medicament, particularly for treatment and/or prophylaxis of IBD.

In a further aspect, the invention refers to the use of the formulation of the invention in the manufacture of a medicament for treatment and/or prophylaxis of IBD.

In a further aspect, the invention refers to a method for treatment and/or prophylaxis of a patient suffering from IBD, or likely to suffer IBD, said method comprising administering to the subject in need of such treatment or prophylaxis a therapeutically effective amount to the pharmaceutical composition described herein.

In view of its systemic anti-inflammatory action, the pharmaceutical formulation of the invention is also useful for treatment and/or prophylaxis of the rheumatic manifestations associated with IBD.

These aspects and preferred embodiments thereof are additionally also defined in the detailed description as well as in the claims.

### FIGURES

FIG. 1: In vitro release profiles of Example 1 and Example 2 compositions

### DETAILED DESCRIPTION OF THE INVENTION

The technical problem underlying the present invention is to provide an alternative Mesalamine composition that offers greater variety and flexibility in the release profile to be obtained. The composition of the present invention allows the release of Mesalamine through the entire gastrointestinal tract as well as it ensures a systemic absorption of Mesalamine.

The formulation of the present invention surprisingly controls the release of Mesalamine through the small and large intestines and, at the same time provides a variable amount of Mesalamine which is available to be absorbed systemically under the acidic pH conditions of the stomach. The formulation of the present invention may be useful to treat patients suffering from IBD and presenting extraintestinal manifestations such as rheumatism. Moreover, another advantage of the present formulation is that by modulating process and formulation parameters, the percentage of Mesalamine available in the acidic pH environment of the stomach and, thus with a probability to be absorbed and to have a systemic action, could be modified to target a specific group of patients suffering from. Examples of formulation and process parameters are inert core size, Mesalamine particle size, the type and content of waxy material and/or the hydrophilic polymer and the thickness of the layer.

Moreover, the formulation of the invention presents the advantage that it is cost effective since only one layer is needed to control de release of the active ingredient through the gastrointestinal tract, thus facilitating the manufacture of the final dosage form while achieving a control on the release of the active ingredient. For example, the formulation of the invention is free from any intermediate layer between the inner core and the controlled release layer and, typically, it is also free from an enteric coating. As enteric coating has to be understood enteric coating polymers used separately or in combination, in the form of solutions or dispersions, those polymers being in particular chosen from methacrylic acid copolymers, polysorbates, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac, or other suitable enteric polymer(s) which can be formulated with other well known excipients in the art to form the enteric coating.

In addition to the optimum dissolution profile, the formulation of the invention presents the advantage of exhibiting excellent storage stability.

The composition of the present invention is a controlled release pharmaceutical formulation. This means that the release of the active ingredient, Mesalamine, from the formulation is prolonged and/or sustained so that, the time between administered doses is increased.

By the term “formulation in solid form” it has to be understood a preparation—medicament or solid dosage form—in solid state such as a pellet, granule, tablet, capsule, minitablet, sachet, stick pack, etc. which comprises Mesalamine to be released to an appropriate medium such as gastrointestinal fluid.

By “pharmaceutically acceptable” such as in the recitation of a “pharmaceutically acceptable excipient” is meant herein a material that is not biologically or otherwise undesirable, i.e., the material may be incorporated into a pharmaceutical composition administered to a patient without causing any undesirable biological effects or interacting in a deleterious manner with any of the other components of the composition in which it is contained.

As used herein, the term “about” means a slight variation of the value specified, preferably within 10 percent of the value specified. Nevertheless, the term “about” can mean a higher tolerance of variation depending on for instance the experimental technique used. Said variations of a specified value are understood by the skilled person and are within the context of the present invention. Further, to provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

Unless otherwise stated, all amounts are expressed herein as percentage by weight in a dry matter basis.

The formulation in solid form of the invention comprises:

a) an immediate release inner core comprising Mesalamine and a pharmaceutically acceptable carrier, and

b) a controlled release layer comprising a mixture of a waxy material and a hydrophilic polymer, said layer being directly disposed onto the inner core.

The drug release rate of Mesalamine from the formulation of the invention shows a specific controlled release behavior. The controlled release layer of the formulation of the invention enhances Mesalamine solubility at pH 1.2 which is representative of stomach environment. This result is totally unexpectedly based on the thermodynamic solubility of Mesalamine which is 7 folds higher at pH 7.5 than at pH 1.2.

According to particular embodiments, said dosage form is a particle, granule, a pellet, a minitablet or a tablet.

In a preferred aspect, the invention is directed to a capsule or a tablet or a sachet or a stick pack or a pouch comprising a plurality of the aforementioned particles, granules, pellets, minitablets or tablets. Preferably, the invention is directed to a capsule.

### The Inner Core

By the term inner core is understood a granule, pellet or minitablet which comprises the active ingredient and a pharmaceutically acceptable excipient or mixtures thereof. Examples of pharmaceutically acceptable excipients that may be comprised in the inner core are fillers, binders, disintegrants and lubricants. These excipients, together with Mesalamine, may be used alone or in any possible combination thereof.

According to the invention, the inner core is an immediate release inner core which has to be understood as having a release of at least about 80% of the Mesalamine within 15 minutes in HCl 0.1N pH 1.0, preferably a release of at least about 80% of the said drug within 10 minutes in HCl 0.1N pH 1.0, and more preferably a release of at least about 80% of the said drug within 5 minutes in HCl 0.1N pH 1.0, with USP (II) paddles under the following conditions 100 rpm and volume 900 ml.

For the purposes of the invention, “granule” is understood as a solid form which is spheroidal and manufactured by methods of granulation. The granule comprises Mesalamine and one or more pharmaceutical excipients preferably selected from the group of binders, fillers and lubricants. There are different methods for preparing granules such as wet granulation and dry granulation. These methods of granulation include the use of conventional granulators e.g. spray granulators, rotary granulators, centrifugal fluidized bed granulators, high-speed mixer granulators. A related granulation process is the spheronization which refers to the formation of spherical particles form wet granulations. In this method, the wet granules are then extruded and subjected to a process of spheronization. Drying may be carried out by conventional techniques, for example in the granulator or in a drying oven or hot air drier. It is, of course, desirable that the granules should be prepared by a method, which is convenient to provide granules of the desired size and shape; this may generally be achieved by conventional adjustment of the conditions of granulation, or optionally by adjustment of extrusion-spheronization process. Furthermore, if necessary, the desired size and shape is provided by screening of the granules thus produced. The resulting granules are then dried in a fluid bed dryer and then screened to get the granules of the desired shape and size.

For the purposes of the invention “pellet” is understood as solid form manufactured in multilayer form and having a core-sheath structure. The internal core of the pellet may be formed by an inert bead which is then covered with a layer comprising the Mesalamine together with appropriate pharmaceutical excipients well known in the art. The internal bead is inert with regard both to active ingredient, Mesalamine, and to the other excipients in the pellet, and with regard to the patient who will ingest the pellet. Such inert bead is conventionally used in pharmaceutical techniques. The bead may be prepared from materials such as, e. g. starch, sucrose, cellulose, microcrystalline cellulose, carnauba wax, tartaric acid, silicon dioxide and the like. The size of the beads depends on the desired size of the pellet to be manufactured or further processed and on the desired amount of Mesalamine to be dosed per gram of pellet. Typically, the bead represents from about 20% to 60% by weight of the pellet.

For the purposes of the invention “minitablet” is understood as solid form manufactured by compression of the active ingredient (e.g. Mesalamine) together with pharmaceutical acceptable excipients such as fillers, binders, lubricants, disintegrants and the like, alone or in combination. They have usually a size ranging from about 1 mm to 3 mm in diameter.

In a preferred embodiment, the inner core is a granule prepared by an extrusion-spheronization process. These granules present the advantage of allowing a high drug loading.

The amount of Mesalamine in the inner core may vary for example from about 50% to 95% with respect to the total weight of the dry matter of the formulation. In particular, amounts of about 65% to 95%, preferably of about 72 to 92% have been found to be appropriate. The inner core containing the active ingredient may include excipients commonly used in pharmaceutical formulations that do not interact adversely with Mesalamine. As indicated before, examples of these excipients that can be used alone or in combination are fillers, binders, lubricants, disintegrants and the like. In a particular embodiment, the inner core comprises the following excipients: a binder, a filler and a lubricant.

Examples of fillers include, but are not limited to, sucrose, glucose, lactose, mannitol, xylitol, dextrose, microcrystalline cellulose, coprocessed microcrystalline celluloses (such as Avicel CL-611, Avicel RC-581, Avicel RC591, Avicel CE, Avicel DG, Avicel HFE), maltose, sorbitol, calcium phosphate, calcium sulphate, carrageenan, chitosan, pectinic acid, sodium alginate, magnesium aluminium silicate and the like can be given. Preferably, the filler is microcrystalline cellulose.

The preferred percentage of filler in the inner core according to this invention is from about 0.1% to about 50%, preferably about 0.5% to 20%, more preferably about 1% to 15% by weight with respect to the total weight of the dry matter of the formulation.

Examples of binders include, but are not limited to, celluloses such as microcrystalline cellulose, modified celluloses (such as low substituted hydroxypropyl cellulose, hydroxypropyl cellulose (or HPC), hydroxypropyl methylcellulose (or HPMC or hypromellose), hydroxyethylcellulose, hydroxyethyl methylcellulose, ethyl cellulose, cellulose gum, xanthan gum, sugars (such as sucrose, glucose, amilose, maltodextrin, dextrose and the like), starches such as corn or potato starch partially pregelatinized starches (such as Starch 1500), polyvinyl acetate (Kollicoat SR), polyvinyl alcohol-polyethylene glycol graft copolymer (Kollicoat IR), copovidone, cross-linked polyvinylpyrrolidone, acrylic acid polymer (Carbopol), poloxamer, polycarbophil, polyethylene oxide, polyethylene glycol or a combination thereof. Preferably, the binder is corn starch.

The preferred percentage of binder in the inner core according to this invention is from about 0.1% to about 30%, preferably about 0.1% to 10%, more preferably about 0.1% to 5% by weight with respect to the total weight of the dry matter of the formulation.

The following are examples of useful disintegrants: starches such as corn or potato starch, modified starches (such as sodium starch glycolate) and partially pregelatinized starches (such as Starch 1500); polyvinylpyrrolidones, including modified polyvinylpyrrolidones (such as crospovidone, polymerized under conditions that promote crosslinking), crosslinked carboxymethylcellulose sodium (cross carmellose sodium), ion exange resins (such as Polacrilin potassium, Polacrilex) Neusilins, low substituted hydroxypropyl cellulose or a combination thereof.

The preferred percentage of disintegrant in the inner core according to this invention is from about 0.1% to about 20%, preferably about 1% and 18%, more preferably about 5 to 15% by weight with respect to the total weight of the dry matter of the formulation.

Examples of lubricants include, but are not limited to, calcium stearate, glyceryl monostearate, glyceryl palmitostearate, magnesium stearate, sodium stearyl fumarate, talc powder, colloidal silicon dioxide or the mixture.

The preferred percentage of lubricant in the inner core according to this invention is from about 0.5% to about 10% by weight with respect to the total weight of dry matter of the formulation. The most preferred percentage is about 1.0% to 7.0% by weight with respect to the total weight of dry matter of the formulation.

### The Controlled Release Layer

The controlled release layer used in the controlled release oral pharmaceutical formulation in solid form of the invention comprises a mixture of a waxy material and a hydrophilic polymer. This controlled release layer is directly disposed onto the inner core which means that no additional layers exist between the inner core and the controlled release layer.

In a preferred embodiment of the controlled release layer used in the formulation in solid form according to the invention, the waxy material is selected from hydrophobic waxy materials to provide a controlled release of the active agent over the entire small and large intestines. For the purposes of the invention, waxy materials are preferably selected from materials such as camauba wax, white wax, candelilla wax, beeswax, cetylester wax, montan wax, microcrystalline wax, lecithin, hydrogenated tallow, paraffin wax, sellac wax, paraffin soft, glyceril behenate, glycerol palmitoesterarate, glycerol diestearate, tribehenin, glycerol esters such as glyceril behenate and glyceryl palmitostearate, fatty alcohols (particularly those having 12-24 carbon atoms, such as cetyl alcohol, cetostearyl alcohol, lauryl alcohol, myristyl alcohol, stearyl alcohol, palmityl alcohol, etc), fatty acids (particularly those having 12-24 carbon atoms, such as lauric acid, myristic acid, stearic acid, palmitic acid, etc), castor wax (i.e. hydrogenated castor-oil), C₁₆₋₃₀ fatty acid triglycerides stearyl polyoxil-32 glyceride, behenoyl polyoxil-8 glyceride, lauroyl polyoxil glycerides, steral polyoxil glycerides, and mixtures thereof. Preferably, the waxy material is selected from white wax, glyceril behenate, glycerol palmitoesterarate and fatty alcohols such as cetyl alcohol, cetostearyl alcohol, lauryl alcohol, myristyl alcohol, stearyl alcohol and palmityl alcohol. The most preferred waxy material is glyceril behenate and among the fatty alcohols, the most preferred is cetyl alcohol.

In another preferred embodiment of the controlled release layer used in the formulation according to the invention, the hydrophilic polymer is selected from polyvinyl alcohol, sodium carboxy methylcellulose, hydroxypropylcellulose, hydroxypropylmethyl cellulose, hydroxyethylmethylcellulose, polyvinylpyrrolidone, copovidone starch or its derivates, sodium alginate, calcium alginate, sodium carboxymethylcellulose, calcium carboxymethylcellulose, polyethylene glycol (PEG) having a molecular weight of greater than 1000 number average molecular weight (e.g. PEG 3350, PEG 4600, PEG 6000, PEG 8000, PEG 12000 and PEG 20000) polyethylene oxide or the mixtures thereof. Preferably, the hydrophilic polymer is hydroxypropylmethyl cellulose.

Preferably, the controlled release layer is free from ethylcellulose.

As indicated before, the release achieved is not targeted in a specific pH medium and thus, the controlled release layer of the present invention may be free from enteric polymers chosen from methacrylic acid copolymers (Eudragit L, RS, RL, NE), polysorbates, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or the mixtures thereof.

Other excipients may also be present in the controlled release layer of the oral formulation according to the invention such as surfactants and lubricants, which can also act as plasticizers. These excipients can be used alone or in combination in the layer together with the hydrophilic polymer and the waxy material describe above.

Surfactants to be used in the controlled release layer used in the formulation of the invention may be selected from the non-limiting list of materials which includes sodium dodecylsulfate, polysorbate 80, polysorbate 20, polysorbate 40, polysorbate 60, polysorbate 65, polysorbate 85, polysorbate 80, polysorbate 21, polysorbate 61, poloxamer 188, macrogolglycerol ricinoleate and mixtures thereof. In some other preferred aspects of the invention, the most preferred surfactant is polysorbate 80.

The preferred percentage of surfactant is about 0.1 to 10% by weight with respect to the total weight of dry matter of the formulation. The most preferred percentage is about 0.15 to 5.0% by weight with respect to the total weight of dry matter of the formulation.

In a preferred embodiment, the controlled release layer used in the formulation according to the invention comprises a mixture of a waxy material, a hydrophilic polymer and at least one surfactant.

The amount of surfactant is preferably below 5% by weight with respect to the waxy material and more preferably, between 1 and 4% by weight with respect to the waxy material.

In another preferred embodiment, the controlled release layer used in the formulation according to the invention further comprises a lubricant. The lubricants are selected from at least one of the following components: calcium stearate, glyceryl monostearate, glyceryl palmitostearate, magnesium stearate, sodium stearyl fumarate, talc powder, colloidal silicon dioxide or the mixture thereof.

The preferred percentage of lubricant in the controlled release layer used in the formulation according to the present invention is about 0.1 to 10% by weight with respect to the total weight of dry matter of the formulation. The most preferred percentage is about 0.5% to 5.0% by weight with respect to the total weight of dry matter of the formulation.

In a preferred embodiment of the controlled release layer used in the formulation according to the invention the weight ratio of the waxy material and the hydrophilic polymer (Ratio=waxy material/hydrophilic polymer) is between about 20 and 0.5. In another preferred embodiment, the weight ratio of the waxy material and the hydrophilic polymer is between about 15 and 1, preferably between about 10 and 1 and most preferably between about 6 and 1.5.

In a further preferred embodiment, the controlled release layer is free from Mesalamine.

In other preferred embodiment, the controlled release layer used in the formulation of the invention is an aqueous dispersion comprising the waxy material and the hydrophilic polymer. Optionally, other excipients as those mentioned above can also be present in the dispersion alone or in combination. This aqueous dispersion may be applied directly onto the inner core using well known techniques in the art.

According to a preferred embodiment, the controlled release layer used in the formulation of the invention consists of a waxy material, a hydrophilic polymer, a surfactant and a lubricant. Preferably, the waxy material is cetyl alcohol or glyceryl behenate, the hydrophilic polymer is hydroxypropylmethyl cellulose, the surfactant is polysorbate 80 and the lubricant is glyceryl monoestearate.

### The Final Formulation

The final formulation of the present invention may be a particle, granule, a pellet, a minitablet, a capsule or a tablet or a sachet or a pouch or a stick pack. Preferably, the formulation is a capsule or a tablet comprising a plurality of particles, granules, pellets, minitablets or tablets according to the invention.

In addition, the formulation according to the invention may comprise other substances commonly used in the art, for example, at least one additive selected from the group consisting of colorants, flavour masking agents, flavouring agents, stabilizers, fillers, antifoaming agents, sweeteners, pore-forming agents, pH modifying agents, antioxidants, pigments and the like.

As examples of the masking agent, water insoluble polymers such as ethyl cellulose, polymers insoluble in saliva and soluble in gastric fluid such as a copolymer of methyl methacrylate, butyl methacrylate, and diethylaminoethyl methacrylate, and the like can be given.

As examples of flavouring, perfume, lemon, lemon-lime, orange, menthol, peppermint oil, vanillin or powders of these absorbed with dextrin or cyclodextrin, and the like can be used.

As examples of the colorant, food dyes such as food yellow No. 5, food red No. 3, food blue No. 2, food lake dye, red iron oxide, and the like can be given.

As examples of the stabilizer or solubilizer, antioxidants such as ascorbic acid and tocopherol, surfactants such as those indicated before and the like can be given depending on the physiologically active component used.

As examples of filler, sucrose, glucose, lactose, mannitol, xylitol, dextrose, microcrystalline cellulose, maltose, sorbitol, calcium phosphate, calcium sulphate and the like can be given.

As examples of the antifoaming agent, dimethicone can be used.

The formulation of the invention can further be coated through processes well known to the persons skilled in the art to provide one or more layers for the purposes of taste masking and ease of further processing. Conventional coating techniques, for example, spray coating using a fluidized bed granulator, a centrifugal fluidized bed coater or a spray drier or coating with a rotary granulator can be used.

In a preferred embodiment, the final formulation is free from enteric polymer. Therefore, preferably, the final formulation of the invention does not comprise any layer related to the drug release profile in addition to the controlled release layer.

In additional preferred embodiments, the preferences described above for the components of the pharmaceutical formulation are combined. The present invention is also directed to such combinations of preferred components within the pharmaceutical formulation.

### Method for the Preparation of the Formulation

In an embodiment, the pharmaceutical compositions of the present invention can be prepared by a process comprising:

a. obtaining Mesalamine inner cores, and

b. coating the Mesalamine inner cores with an aqueous dispersion comprising a mixture of waxy material and hydrophilic polymer.

The Mesalamine inner cores can be formed by any process, such as dry granulation, wet granulation, extrusion-spheronization, compression, and the like. In some preferred embodiments, the granulation, optionally with pharmaceutically acceptable excipients like binders and/or fillers and/or lubricants and/or disintegrants, can be carried out in various apparatus, such as a rapid mixer granulator or fluidized bed processor.

The aqueous dispersion comprising a mixture of waxy material and hydrophilic polymer may be prepared by known methods in the art.

In one embodiment, the process for the preparation of the dispersion comprises:

1. Preparing a dispersion of the waxy material into hot water (preferably, the temperature of the water should be above the melting point of the waxy material), and

2. Adding the hydrophilic polymer under stirring and cool down to room temperature.

A preferred process for the preparation of the dispersion comprises:

1. Preparing a dispersion of the waxy material, a surfactant and/or a lubricant into hot water (preferably, the temperature of the water should be above the melting point of the waxy material), and

2. Adding the hydrophilic polymer under stirring and cool down to room temperature.

In another preferred embodiment, the process comprises preparing a dispersion of all the components together i.e. waxy material, hydrophilic polymer and optionally surfactant and/or lubricant into hot water under stirring and cool down to room temperature. The excipients may be added sequentially, for instance in the following order: Surfactant, lubricant, hydrophilic polymer and waxy material.

The dispersion obtained from the above processes can be screened in order to confirm the absence of any insoluble material and the uniformity of the dispersion.

Finally, the resulting dispersion is sprayed onto the Mesalamine inner cores using a fluid bed processor.

The process used for the preparation of the formulation of the invention is characterized in that it may be free from organic solvents.

The finally obtained granules, pellets or minitablets can be compressed into tablets or filled into capsules or sachets by techniques known in the art. Alternatively, tablets can be prepared by direct compression techniques.

The amount of the dose administered, as well as the dose frequency, will vary depending on the particular dosage form used. The amount and frequency of administration will also vary according to the age, the body weight, and response of the individual subject. Typical dosage regimens can readily be determined by a competent physician. It is also noted that the clinician or treating physician will know how and when to interrupt, adjust or terminate therapy in conjunction with individual subject response.

In a further aspect, the invention refers to the use of the formulation of the invention for treatment and/or prophylaxis of a patient suffering from IBD, comprising administering to the subject in need of such treatment or prophylaxis a therapeutically effective amount to the pharmaceutical composition described therein.

The following examples are merely illustrative of certain embodiments of the invention and cannot be considered as restricting it in any way.

### EXAMPLES

### Example 1

Controlled release capsules comprising Mesalamine were made according to the following composition

| Ingredient | % |
|---|---|
| Mesalamine | 75.18 |
| Binder | 0.85 |
| Filler | 7.25 |
| Lubricant | 1.70 |
| Glyceril behenate | 11.32 |
| Hydroxypropyl | 2.37 |
| methylcellulose | |
| Surfactant | 0.31 |
| Lubricant | 1.02 |
| Total | 100.00 |

The process for the preparation of the formula was as follows:

751.8 g of Mesalamine, 8.5 g of binder, 72.5 g of filler and 17.0 g of lubricant were loaded in a high shear mixer. The components were mixed at low speed to obtain a homogeneous blend. After mixing, purified water was sprayed on the blend until a wet plastic mass was achieved.

The wet mass was forced to pass through a screen extruder. The extruded material was loaded is a spheronizer to obtain spherical granules. Then the granules were dried in a fluid bed dryer.

The granules were selected to obtain the desired size by screening and loaded in a fluid bed coater where they were sprayed with a dispersion obtained by the following process:

1155 g of purified water were heated to 80° C., then 3.1 g of surfactant, 10.2 g of lubricant and 113.2 g of glyceril behenate were dispersed by stirring in the water and homogenized by high shear rotor-stator stirrer, and subsequently 23.7 g of hydroxypropyl methylcellulose were dissolved in the dispersion. The dispersion was cooled down below 30° C. and filtered through a sieve of 400 μm.

The coated granules were dosed in capsules to obtain the desired dose.

### Example 2

Controlled release capsules comprising Mesalamine were made according to the following composition

| Ingredient | % |
|---|---|
| Mesalamine | 70.74 |
| Binder | 0.80 |
| Filler | 6.82 |
| Lubricant | 1.60 |
| Cetyl Alcohol | 12.18 |
| Hydroxypropyl | 6.39 |
| methylcellulose | |
| Surfactant | 0.32 |
| Lubricant | 1.05 |
| Total | 100.00 |

The process for the preparation of the formula was as follows:

707.4 g of Mesalamine, 8.0 g of binder, 68.2 g of filler and 16.0 g of lubricant were loaded in a high shear mixer. The components were mixed at low speed to obtain a homogeneous blend. After mixing, purified water was sprayed on the blend until a wet plastic mass was achieved.

The wet mass was forced to pass through a screen extruder. The extruded material was loaded is a spheronizer to obtain spherical granules. Then the granules were dried in a fluid bed dryer.

The granules were selected to obtain the appropriate size by screening and loaded in a fluid bed coater where they were sprayed with a dispersion obtained by the following process:

1155 g of purified water were heated to 80 PC, then 3.2 g of surfactant, 10.5 g of lubricant and 121.8 g of cetyl alcohol were dispersed by stirring in the water and homogenized by high shear rotor-stator stirrer, and subsequently 63.9 g of hydroxypropyl methylcellulose were dissolved in the dispersion. The dispersion was cooled down below 30° C. and filtered through a sieve of 400 μm.

The coated granules were dosed in capsules to obtain the desired dose.

### Example 3

### Dissolution Profiles

Dissolution profiles of formulations of both examples 1 and 2 were tested in a dissolution method which reflects gastrointestinal transit times. The test is performed with USP dissolution Apparatus II, at a rotation speed of 100 rpm and it is divided in three steps: gastric fluid (acidic dissolution stage, 500 mL of 2 g/L NaCl, pH adjusted to 1.0 with HCl 0.1N) for 2 h; small intestinal fluid (addition of 245 mL of 0.1 M Na₃PO₄.12 H₂O to bring pH adjusted to 6.0) for 2 h; and large intestinal fluid (addition of 100 mL of 0.1 M Na₃PO₄.12 H₂O to raise medium pH to 7.3) for 8 h.

| | TIME (h) | | | | | | | | | |
|---|---|---|---|---|---|---|---|---|---|---|
| | Medium: | | | Medium: | | | | | | |
| % | pH 1.0 | | | pH 6.0 | | Medium: pH 7.3 | | | | |
| Dissolved | 0 | 1 | 2 | 3 | 4 | 4.5 | 5 | 6 | 8 | 12 |
| Ex. 1 | 0.0 | 47.1 | 79.7 | 85.0 | 90.0 | 93.4 | 96.5 | 101.5 | 106.5 | 108.1 |
| Ex. 2 | 0.0 | 59.2 | 81.4 | 86.1 | 90.2 | 92.3 | 94.5 | 98.1 | 103.0 | 103.0 |

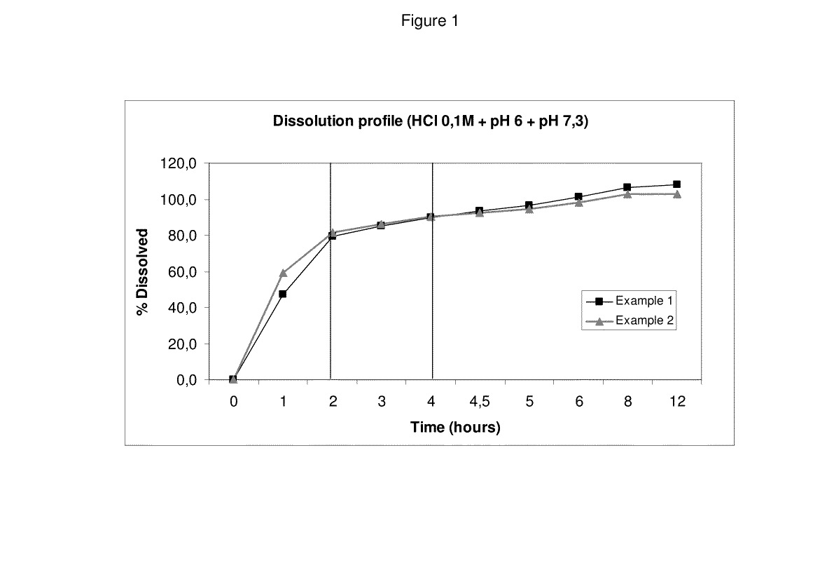

The above table as well as FIG. 1 demonstrates that about 80% of the Mesalamine is released within 2 h in the gastric fluid while the rest of Mesalamine is released, continuously, over the small and large intestines.

1-19. (canceled)

20. A controlled release oral pharmaceutical formulation in solid form comprising:
a) an immediate release inner core comprising Mesalamine and a pharmaceutically acceptable excipient, and
b) a controlled release layer comprising a mixture of a waxy material and a hydrophilic polymer, said layer being directly disposed onto the inner core.

21. The formulation according to claim 20, herein the pharmaceutically acceptable excipient is one at least selected from the group consisting of fillers, binders, disintegrants, lubricants and mixtures thereof.

22. The formulation according to claim 20, wherein the waxy material is selected from camauba wax, white wax, candelilla wax, beeswax, cetylester wax, montan wax, microcrystalline wax, lecithin, hydrogenated tallow, paraffin wax, sellac wax, soft paraffin, tribehenin, glycerol esters, fatty alcohols, fatty acids, castor wax, C₁₆₋₂₀ fatty acid triglycerides, and mixtures thereof.

23. The formulation according to claim 22, wherein the waxy material is selected from white wax, glyceryl behenate, glyceryl palmitoesterarate, cetyl alcohol, cetostearyl alcohol, lauryl alcohol, myristyl alcohol, stearyl alcohol, palmityl alcohol and mixtures thereof.

24. The formulation according to claim 20, wherein the hydrophilic polymer is selected from polyvinyl alcohol, carboxymethylcellulose sodium, hydroxypropyl cellulose, hydroxypropyl methylcellulose, hydroxyethyl methylcellulose, polyvinylpyrrolidone, copovidone, starch or its derivates, sodium alginate, calcium alginate, carboxymethylcellulose sodium, carboxymethylcellulose calcium, polyethylene glycol (PEG) having a molecular weight of greater than 1000 number average molecular weight, polyethylene oxide and the mixtures thereof.

25. The formulation according to claim 20, wherein the controlled release layer further comprises at least a lubricant or at least a surfactant or mixtures thereof.

26. The formulation according to claim 25, wherein the lubricant is selected from calcium stearate, glyceryl monostearate, glyceryl palmitostearate, magnesium stearate, sodium stearyl fumarate, talc powder or the mixtures thereof.

27. The formulation according to claim 25, wherein the surfactant is selected from mono- and diglycerides, medium chain glycerides (Capmul), glyceryl ricinoleate, glyceryl laurate, glyceryl caprylate, PEG sorbitan fatty acid esters, Sorbitan fatty add esters, sugar ester surfactants, ionic surfactants, phospholipids, alginate salts or the mixtures thereof.

28. The formulation according to claim 20, wherein the weight ratio of the waxy material and the hydrophilic polymer is between 20 and 0.5.

29. The formulation according to claim 20, wherein the controlled release layer is free from Mesalamine.

30. The formulation according to claim 20, wherein the formulation in solid form is a particle, granule, a pellet, a minitablet or a tablet.

31. A tablet comprising a variable number of particles, granules, pellets, minitablets or tablets according to claim 30.

32. A capsule comprising a variable number of granules, pellets, minitablets or tablets according to claim 30.

33. A sachet comprising a variable number of particles, granules, pellets, minitablets or tablets according to claim 30.

34. A method for the preparation of the formulation according to claim 20 which comprises
(a) obtaining Mesalamine inner cores,
(b) coating the Mesalamine inner cores with an aqueous dispersion comprising a mixture of waxy material and hydrophilic polymer.

35. The formulation according to claim 22, wherein the waxy material is selected from glyceryl behenate, glyceryl palmitostearate, cetyl alcohol, cetostearyl alcohol, lauryl alcohol, myristyl alcohol, stearyl alcohol, palmityl alcohol, lauric acid, myristic add, stearic acid, palmitic acid, stearyl polyoxyl-32 glyceride, behenoyl polyoxyl-8 glyceride, lauroyl polyoxyl glycerides, and mixtures thereof.

36. The formulation according to claim 27, wherein the surfactant is selected from glyceryl monooleate (Peceol®, PEG-20 sorbitan monolaurate (Tween® 20), PEG-20 sorbitan monoestearate (Tween® 60), PEG sorbitan monooleate (Tween® 80), sorbitan monolaurate (Span® 20), sucrose distearate, sodium caprylate, sodium lauryl sulphate, or the mixtures thereof.

37. The formulation according to claim 26, wherein the weight ratio of the waxy material and the hydrophilic polymer is between 15 and 1.

38. The formulation according to claim 28, wherein the weight ratio of the waxy material and the hydrophilic polymer is between 10 and 1.
